(19) United States
(12) Patent Application Publication
(10) Pub. No.: US 2006/0177355 A1
(43) Pub. Date: Aug. 10, 2006

(54) Combination of a computer casing and an lonizer

(76) Inventor: Chen-Cheng Chien, Tan Zi Hsiang (TW)

Correspondence Address: NIKOLAI & MERSEREAU, P.A., 900 SECOND AVENUE SOUTH, SUITE 820, MINNEAPOLIS, MN, 55402, US

(21) Appl. No.: 11/052,430

(22) Filed: Feb. 7, 2005

(51) Int. Cl. A61L 9/22

(52) U.S. Cl. 422/121

(57) ABSTRACT

A computer casing has multiple compartments defined therein for receiving different electronic accessories and two L shaped supporting plates oppositely and securely placed in one of the compartments. An ionizer is received in one of the compartments to generate negative ions and has holes allowing the negative ions to pass through the holes for charging air surrounding the computer casing.

### BACKGROUND OF THE INVENTION

1. Field of the Invention

The present invention relates to a computer casing, and more particularly to the computer casing having therein an ionizer to generate negative ions to charge the ambient air surrounding the computer user.

2. Description of Related Art

Throughout the world, people are becoming increasingly exposed to computers and now there is almost nothing in the current world that is not related to computers, especially those so-called white collar workers. To improve work conditions, air conditioners are provided such that people may be most productive. However, it is commonly-believed that in an air-conditioned enclosed space, people will gradually develop illnesses which are related to air quality of the enclosed space.

To overcome the shortcomings, the present invention tends to provide an improved computer casing having therein an ionizer to mitigate the aforementioned problems.

### SUMMARY OF THE INVENTION

The primary objective of the present invention is to provide an improved computer casing provided with an ion generator to generate negative ions to charge ambient air surrounding the computer.

Other objects, advantages and novel features of the invention will become more apparent from the following detailed description when taken in conjunction with the accompanying drawings.

### BRIEF DESCRIPTION OF THE DRAWINGS

FIG. 1 is an exploded perspective view showing the internal structure of the ionizer of the present invention;

FIG. 2 is schematic view showing the combination between the computer casing and the ionizer; and

FIG. 3 is a schematic view showing the internal structure relationship between the computer casing and the ionizer.

### DETAILED DESCRIPTION OF THE PREFERRED EMBODIMENT

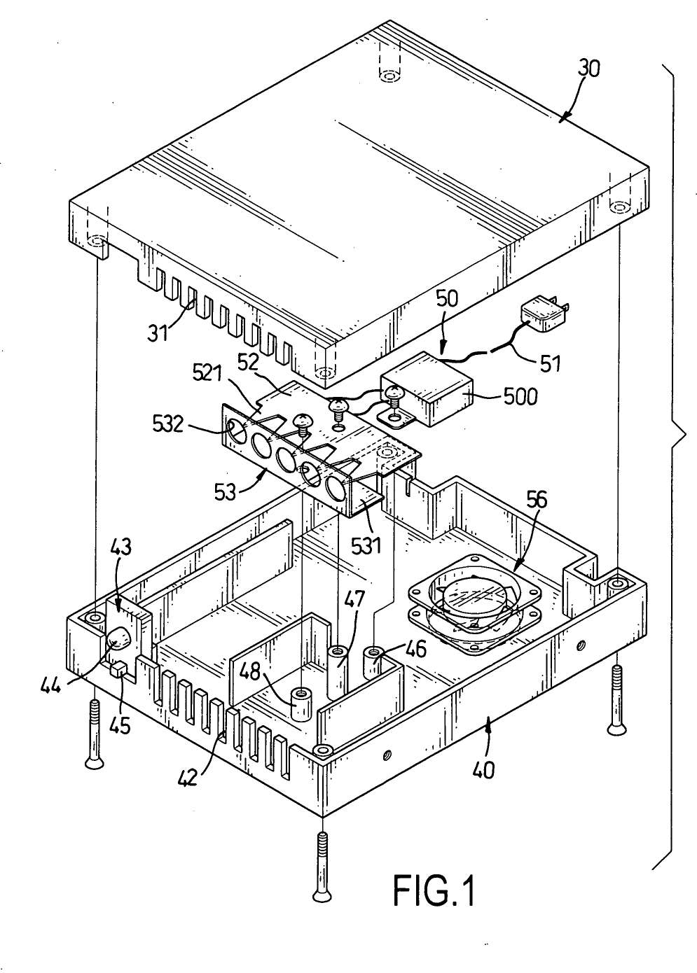

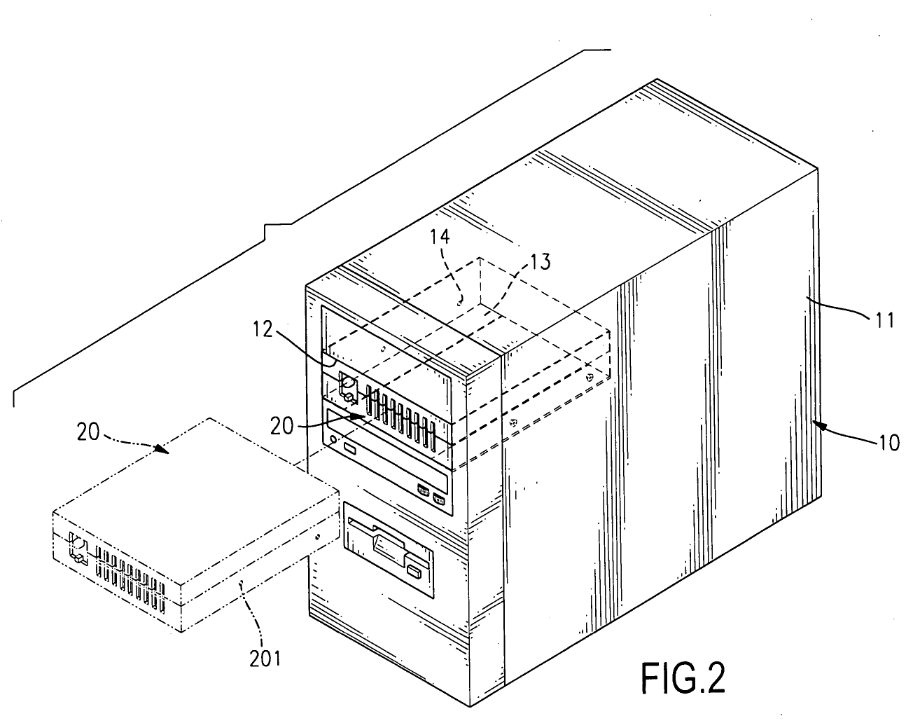

With reference to FIGS. 1 and 2, the ionizer (20) in accordance with the present invention includes a top cap (30) and a bottom cap (40) together with the top cap (30) to define therebetween a receiving space (not numbered), and a high voltage generator (50).

The top cap (30) has multiple first cutouts (31) defined in a side face of the top cap (30). The bottom cap (40) is provided with multiple second cutouts (42) defined in a side face of the bottom cap (40) to correspond to and align with the first cutouts (31) to form multiple holes (not numbered), a control switch (43) sandwiched between the top cap (30) and the bottom cap (40) and having an indicator (44) to indicate on/off status of the power and a master switch (45) to control power supply. A first pole (46), a second pole (47) and a third pole (47) are formed on a face of the bottom cap (40).

The high voltage generator (50) includes a power cord (51) extending out from a body (500) of the high voltage generator (50) for connection with an outlet, wherein the body (500) is fixedly connected to the first pole (46) to electrically connect to the control switch (43), a discharge plate (52) fixedly engaged with the second pole (47), the discharge plate (52) having multiple sharp ends (521) orientated towards the first cutouts (31) and the second cutouts (42) of the top cap (30) and the bottom cap (40) respectively and an L-shaped electrified interface (53) which is fixedly mounted on the third pole (48).

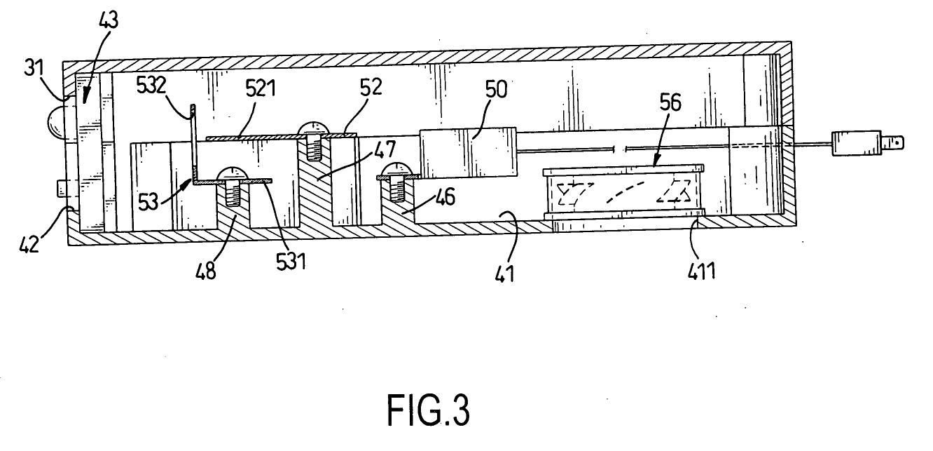

The electrified interface (53) has a horizontal portion used as a ground (531) for connection to the third pole (48) and a vertical portion defining therethrough multiple through holes (532) aligning the multiple sharp ends (521) of the discharge plate (52). A cooling fan (56) is mounted inside the ionizer (20) to blow air toward the high voltage generator (50) for heat dissipation of the high voltage generator (50) and the holes composed of the first cutouts (31) and the second cutouts (42). Furthermore, multiple slits (411) (in FIG. 3) are defined through the bottom cap (40) so that fresh air is able to be drawn into the ionizer (20) by the cooling fan (56).

With reference to FIG. 3 and still using FIG. 2 for reference, after the ionizer (20) is mounted in the computer casing (10) which is provided with multiple compartments (12) for receiving therein different electronic accessories, a pair of L-shaped supporting plates (13) respectively and oppositely mounted in one of the compartments (12) to support the ionizer (20) and each supporting plate (13) having two apertures (14) defined through a vertical portion of the supporting plate (13) to allow extension of fasteners such as bolts. Therefore, after the ionizer (20) is received in the specified compartment (12), fasteners (not shown) are applied to extend through the apertures (14) of each of the two supporting plates (13) to abut two opposed outer faces of the ionizer (20) inside the compartment (12).

When the master switch (45) is actuated to supply power to the high voltage generator (50), negative ions are generated via the sharp ends (521) and blown to the holes of the ionizer by the cooling fan (56), such that the air surrounding the computer casing is charged.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A combination of a computer casing and an ionizer, wherein
the computer casing has multiple compartments defined therein for receiving different electronic accessories and two L shaped supporting plates oppositely and securely placed in one of the compartments,
the ionizer is received in the one of the compartments to generate negative ions and has holes allowing the negative ions to pass through the holes for charging air surrounding the computer casing.

2. The combination as claimed in claim 1, wherein the ionizer has a high voltage generator with a discharge plate securely connected to a bottom cap of the ionizer for generating the negative ions and a cooling fan to blow the negative ions out of the ionizer.

3. The combination as claimed in claim 2, wherein the ionizer further has:
an electrified plate securely connected to the bottom cap of the ionizer and having multiple through holes aligning with the sharp ends of the discharged plate; and
a control switch sandwiched between the bottom cap and a top cap together with the bottom cap to define therebetween a receiving space to receive the high voltage generator, the discharge plate, the electrified interface and the cooling fan, the control switch having a master switch for power supply control and an indicator for indication of power status.

4. The combination as claimed in claim 3, wherein the bottom cap has a first pole to fixedly connect to a body of the high voltage generator, a second pole to fixedly connect to the discharge plate and a third pole to fixedly connect to the electrified interface.

5. The combination as claimed in claim 4, wherein each of the holes of the ionizer is composed of a first cutout defined in a side face of the top cap and a second cutout defined in a side face of the bottom cap to correspond to the first cutout of the top cap.
